(19) United States
(12) Patent Application Publication
(10) Pub. No.: US 2010/0241160 A1
(43) Pub. Date: Sep. 23, 2010

(54) METHOD AND KIT FOR CYST ASPIRATION AND TREATMENT

(76) Inventor: Kieran Murphy, Toronto (CA)

Correspondence Address: CLARK & BRODY, 1700 Diagonal Road, Suite 510, Alexandria, VA, 22314, US

(21) Appl. No.: 12/664,081

(22) PCT Filed: Jun. 10, 2008
(86) PCT No.: PCT/US2008/007212

§ 371 Date: Jun. 4, 2010

Related U.S. Application Data
(60) Provisional application No. 60/943,149, filed on Jun. 11, 2007.

(51) Int. Cl. A61M 1/00; A61B 17/03

(52) U.S. Cl. 606/213; 604/319

(57) ABSTRACT

A method and kit for aspirating and treating a cyst is provided. In an embodiment, the method comprises aspirating fluid from the cyst through a first lumen, while venting the cyst using a second lumen, so as to stabilize pressure variations within the cyst. The aspirated cyst is then injected with a tissue sealant using the second lumen, while continuing to vent the cyst using the first lumen. To enhance visualization of the tissue sealant during imaging, the tissue sealant may be augmented so as to increase its radio-opacity.

### FIELD

The present specification relates to a two-needle cyst aspiration and treatment method and associated kit.

### BACKGROUND

Meningeal or perineural (Tarlov) cysts are meningeal dilations of the posterior spinal nerve root sheath most commonly seen at the sacral level. Tarlov cysts can cause progressive radiculopathy, pelvic pain, sphincter dysfunction, and buttock pain. They are most commonly diagnosed by lumbosacral Magnetic Resonance (MR) imaging, and can often be demonstrated by CT myelography to communicate with the spinal subarachnoid space. Narrow necked cysts can enlarge via a net inflow of cerebrospinal fluid through a valve-like mechanism, eventually causing symptoms by distorting, compressing, or stretching adjacent nerve roots. These cysts may be large and expand the spinal canal, sometimes causing erosion of the overlying bone.

Tarlov Cysts patients are complex to manage and have innumerable complaints. There is growing evidence to suggest that Tarlov Cysts can be symptomatic. Studies have suggested that wide necked Tarlov Cysts are generally asymptomatic, while narrow necked cysts are generally symptomatic. Wide and narrow necked cysts can be differentiated based on their T2 signal on Magnetic Resonance Imaging (MRI). Wide necked cysts have the same signal as the general cerebral spinal fluid (CSF) space around the cord, while narrow necked cysts have a higher signal than the adjacent CSF space. Davis et al. (S W Davis, L M Levy, D J LeBihan, S Rajan, D Schellinger. Sacral Meningeal Cysts: Evaluation of MR Imaging. Radiology 1993; 187:445-448) published evidence of signal change within symptomatic cysts. They demonstrated that of 19 patients with 24 cysts, those with narrow necked cysts were consistently symptomatic by comparison to those with wide necked cysts.

Percutaneous treatment of Tarlov cysts using a single-needle approach and Fibrin adhesive injection has been reported by Patel et al. (Patel M, Louie W, Rachlin J. Percutaneous fibrin glue therapy of meningeal cysts of the sacral spine. AJR 168 February 1997). This procedure generally consists of three stages: a) cyst entry and aspiration, b) contrast injection to ensure there is no wide connection between the cyst and the thecal sac, and c) tissue adhesive injection. This standard technique has been known to result in considerable pain, and an unacceptably high incidence of post-operative aseptic meningitis. The pain was related to pressure variation within the cyst cavity, and traction on the dural lining, caused during the aspiration and injection phases of the procedure. Additional examples of single needle injection of fibrin into cysts include:
Davis S W, Levy L M, LeBihan D J, Rajan S, Schellinger D. Sacral meningeal cysts: evaluation of MR imaging. Radiology 1993, 187:445-448;
Ahn N, Sponsellar P, Ahn U, Nallamshetty L, Kuszyk B, Zinireich S J. Dural Ectasia is associated with back pain in Marfan syndrome. Spine 2000. 25 (12):1562-1568; and
Acosta F L Jr, Quinones-Hinojosa A, Schmidt M H, Weinstein P R. Diagnosis and management of sacral Tarlov cysts. Case report and review of the literature. Neurosurg Focus, 2003 Aug. 15; 15(2):E15.

### SUMMARY

According to a first broad aspect of an embodiment, provided a method for aspirating and treating a cyst comprising:

piercing a cyst with a first needle assembly until a distal tip of said first needle assembly is positioned in the region of the apex of the cyst, said first needle assembly comprising a first trocar and a first stylet;

piercing the cyst with a second needle assembly until a distal tip of said second needle assembly is positioned towards the far side of the cyst generally opposite said apex, said second needle assembly comprising a second trocar and a second stylet;

removing said first and second stylets and aspirating the contents of the cyst through said second trocar, said first trocar providing a vent to neutralize pressure variations within said cyst;

injecting a tissue sealant into the aspirated cyst through said second trocar.

In some embodiments, at least one of said first needle assembly and said second needle assembly is comprised of a substantially radiolucent material.

In some embodiments, at least one of said first needle assembly and said second needle assembly is comprised of carbon fibre.

In some embodiments, at least one of said needle assemblies is maintained in position using a needle holder.

In some embodiments, said tissue sealant is augmented so as to increase its radio-opacity during imaging.

In some embodiments, said tissue sealant is augmented with at least one of tantalum, tungsten and iodine to increase its radio-opacity during imaging.

According to another aspect there is provided a method of aspirating and treating a cyst comprising:

aspirating fluid from the cyst through a first lumen;

venting the cyst using a second lumen, so as to stabilize pressure variations within the cyst; and

injecting a tissue sealant into the aspirated cyst using the second lumen, while continuing to vent the cyst using said first lumen.

In some embodiments, said first lumen is provided by a first needle assembly, and wherein said second lumen is provided by a second needle assembly.

In some embodiments, said first lumen and said second lumen are provided on a single instrument.

In some embodiments, said first lumen and said second lumen are provided in a side-by-side arrangement, and wherein a distal opening of said first lumen is positioned approximately 0.5 cm to 3.0 cm above a distal opening of said second lumen.

In some embodiments, said first lumen and said second lumen are provided in co-axial arrangement, wherein said second lumen is defined by a needle that passes through said first lumen, such that said second lumen takes on a generally annular configuration.

In some embodiments, at least one of said first and second lumens are defined by substantially radio-lucent material.

In some embodiments, at least one of said first and second lumens are defined by respective first and second needle assemblies comprised of carbon fibre.

In some embodiments, said tissue sealant is augmented to increase its radio-opacity during imaging.

In some embodiments, said tissue sealant is augmented with at least one of tantalum, tungsten and iodine to increase its radio-opacity during imaging.

According to a further aspect there is provided a kit of parts for cyst aspiration and treatment, said kit comprising:
a first needle assembly comprising a first trocar and a first stylet, a contiguous piercing tip for piercing through a patient's anatomy being formed when said stylet is assembled with said first trocar;
a second needle assembly comprising a second trocar and a second stylet, a contiguous piercing tip for piercing through a patient's anatomy being formed when said stylet is assembled with said second trocar;
a tissue sealant for injection into an aspirated cyst.

In some embodiments, said kit further comprises at least one needle holder for securing one of said first and second needle assemblies in position during piercing and subsequent use.

In some embodiments, at least one of said first needle assembly and said second needle assembly is comprised of a radio-lucent material

In some embodiments, at least one of said first needle assembly and said second needle assembly is comprised of carbon fibre.

In some embodiments, at least one of said needle assemblies is maintained in position using a needle holder.

In some embodiments, said tissue sealant is augmented so as to increase its radio-opacity during imaging.

In some embodiments, said tissue sealant is augmented with at least one of tantalum, tungsten and iodine to increase its radio-opacity during imaging.

### DESCRIPTION OF THE FIGURES

Embodiments will now be discussed, by way of example only, with reference to the attached Figures, in which:

FIG. 1 is a skeletal representation of a human sacrum showing a Tarlov cyst defect in the region of the sacral spinal canal;

FIG. 2 shows a kit of apparatuses for use in cyst aspiration and treatment in accordance with an embodiment;

FIG. 3 shows placement of a first needle assembly into the Tarlov cyst defect as presented in FIG. 1;

FIG. 4 shows placement of a second needle assembly into the Tarlov cyst defect as presented in FIG. 1;

FIG. 5 shows the removal of stylets from respective first and second needle assemblies;

FIG. 6 shows the aspiration of the Tarlov cyst defect through the second needle assembly;

FIG. 7 shows the injection of tissue sealant into the aspirated Tarlov cyst through the second needle assembly;

FIG. 8 shows the Tarlov cyst following aspiration and filling with tissue sealant;

FIG. 9 is an alternate embodiment showing a double-lumen needle for use in the Tarlov cyst aspiration/treatment method;

FIG. 10 is a cross-sectional view of the double-lumen needle showing the double-lumens of semi-circular configuration;

FIG. 11 shows the insertion/placement of stylets into the double-lumen needle of FIG. 10.

FIG. 12 shows the insertion of the double-lumen needle of FIG. 10 into a Tarlov cyst in a human sacrum;

FIG. 13 is a further alternate embodiment showing a vent-hole configured needle assembly for use in the Tarlov cyst aspiration/treatment method;

FIGS. 14a, 14b and 14c show a sample of alternate configurations for the proximal end of the needle assembly;

FIGS. 15a and 15b show a further alternate embodiment in which the first and second lumen are co-axially arranged;

FIG. 16 shows the placement of the needle assembly of FIGS. 15a/15b into the Tarlov cyst defect as presented in FIG. 1;

FIG. 17 shows the removal of the stylet from the needle assembly of FIGS. 15a/15b during treatment of the Tarlov cyst defect as presented in FIG. 1;

FIG. 18 shows the co-axial alignment of a small gauge needle in the trocar of the needle assembly of FIGS. 15a/15b, during treatment of the Tarlov cyst defect as presented in FIG. 1;

FIG. 19 shows the aspiration of the cyst using the needle assembly of FIGS. 15a/15b;

FIG. 20 shows the injection of tissue sealant into the cyst using the needle assembly of FIGS. 15a/15b; and

FIG. 21 shows an alternate embodiment of a double-lumen needle assembly showing a filter positioned at the proximal end of the first lumen.

### DESCRIPTION

The following discussion is largely framed within the aspiration/treatment of Tarlov Cysts, but one skilled in the art will appreciate that the two-needle approach described below would be applicable to any hollow viscus or joint space, whether it be a gallbladder, an abscess, an intracranial ventricle, a Tarlov Cyst or an arthrogram.

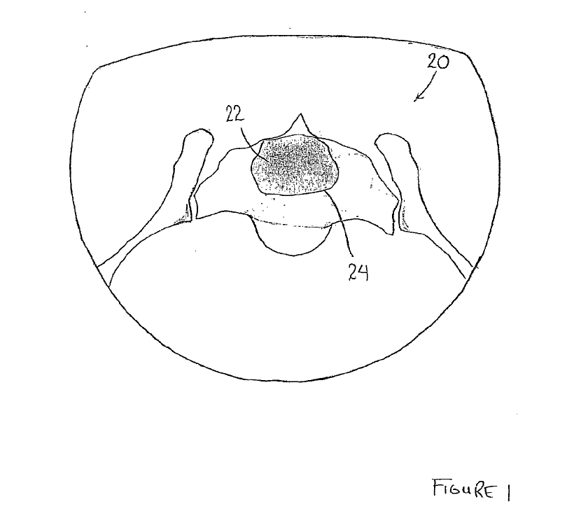

Referring now to FIG. 1, shown is a skeletal representation of a human sacrum as indicated generally by reference numeral 20. As shown, the anatomy presents a Tarlov Cyst 22 defect in the region of the sacral spinal canal 24.

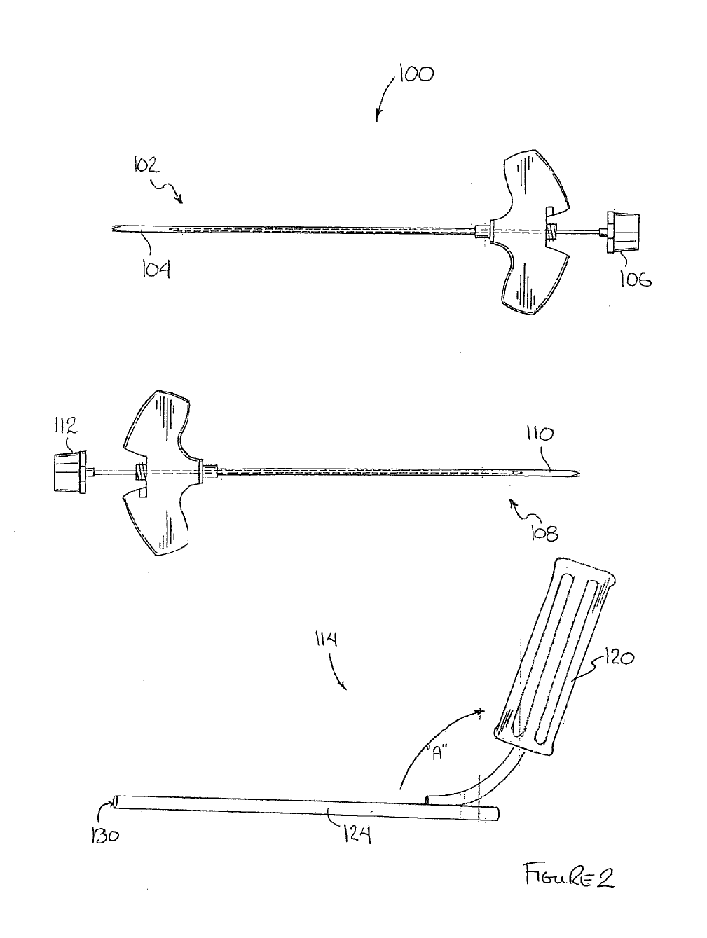

In one embodiment, a method for aspiration and treatment of a Tarlov Cyst is provided. The method can be performed using a kit 100 of apparatuses, an example of which is shown in FIG. 2.

The kit 100 comprises a first needle assembly 102, which itself comprises a hollow trocar 104 and a first stylet 106 that is co-axially received through the hollow trocar 104. The kit further comprises a second needle assembly 108, which itself comprises a hollow trocar 110 and a second stylet 112 what is co-axially received through the hollow trocar 110. When the first needle assembly 102 is assembled, the distal end of the trocar 104 and the tip of the first stylet 106 form a contiguous tip for piercing through tissue. The contiguous tip can take on any variety of configurations, such as a three-sided point, a round arrow-head, or any other suitable arrangement suitable for piercing the intended tissue. The second needle assembly 108 is similarly assembled. Once the piercing and positioning of the needle assembly into the patients anatomy is complete, the stylets 106, 112 can be removed to present the hollow trocars 104, 110 of each needle assembly 102, 108.

The needle assemblies 102, 108 can be any desired gauge, for example any gauge from 12 gauge through to 24 gauge, but is presently preferably 18 gauge. The kit can also contain a needle holder 114 for maintaining the needle assemblies 102, 108 in position during manipulation. Needle holder 114 is typically made of a plastic or other radiolucent material that does not appear under CT image guidance (or under the imaging beam of the particular imaging machine being used). Holder 114 is comprised of a handle portion 120 and a channel portion 124. In a present embodiment, handle portion 120 depends from channel portion at an angle “A” greater than about ninety degrees, however, handle portion 120 can actually depend from channel portion 124 at ninety-degrees or any other desired angle, depending on the procedure being performed, and the preferences of the surgeon or other medical professional performing the procedure. In a present embodiment, handle portion 120 is substantially cylindrical, but can be any desired shape and length, again depending on the preferences and/or needs of the procedure and/or surgeon. Channel portion 124 is also substantially cylindrical, but is further characterized by a hollow channel 130 through which one of the needle assemblies 102, 108 can be passed, and it is presently preferred the hollow channel 130 is of a slightly larger diameter than the needle assemblies to securely hold the needle assembly within the channel portion 124.

In addition to the apparatuses described above, the kit can further comprise components such as, but not limited to, drapes, disinfectants, bandages, anaesthetics (e.g. lidocaine), and syringes.

The cyst aspirating and treatment method explained below is carried out under Computer Tomography (CT) guided fluoroscopy or other suitable imaging system. A presently preferred CT machine for use in the present embodiment is an imaging machine capable of generating substantially real time images. A particularly preferred CT machine for use in conjunction with the method described below is the Toshiba Acquillion, or any other CT machine capable of generating a sufficiently high frame rate to allow real time image generation during a surgical navigation through a patient.

To facilitate CT guidance, the components of the kit 100 are made from a material that is hard enough and/or rigid enough to effect the desired piercing and travelling through tissue, but is also made from a substantially radiolucent material with a radioopacity density such that the components are substantially transparent and create a reduced level of beam hardening artefacts when placed in the imaging beam. In general, the components of the kit have a density that renders them substantially transparent when viewed under the imaging beam. As will be appreciated by those of skill in the art, the presence or absence of a beam hardening artefact can be measured according to the properties of the imaging system being used and in relation to the Hounsfield units associated with the particular material or tissue being exposed to the imaging beam, wherein a lower Hounsfield unit represents a reduced artefact effect when placed under an imaging beam. A relation between the linear attenuation coefficient (μ) and the corresponding Hounsfield unit (H) can be expressed as:

$$H = \frac{μ Material - μ Water}{μ Water} \times 1000$$

The value of the Hounsfield unit varies from −1000 (for air) to 1000 (for bone) to 3000, as more particularly shown in Table I.

TABLE 1
Tissue Range of Hounsfield Units

| Material | Hounsfield Unit |
|---|---|
| Air | −1000 |
| Lung | −500 to −200 |
| Fat | −200 to −50 |
| Water | 0 |
| Blood | 25 |
| Muscle | 25 to 40 |
| Bone | 200 to 1000 |

The foregoing equation and table is found in Principles of Computerized Tomographic Imaging Parallel CT, Fanbeam CT, Helical CT and Multislice CT by Marjolein van der Glas, Aug. 29, 2000, http://www.ph.tn.tudelft.nl/.ab- out.marlein/pdf/ct.pdf

With the various components being manufactured from a substantially radiolucent material, they do not create (or only create suitably reduced) artefacts on the display of the CT machine during their real-time display during use. Suitable materials can include, but are not limited to, certain plastics (e.g. Polyetheretherketones (PEEK)), carbon fiber and Inconel metals. While a rate of image display in “real-time”, such as about 13 frames per second (“fps”) or greater is presently preferred, other rates can also be chosen, such as greater than about 20 fps, or greater than about 25 fps, or greater than about 30 fps. It is also to be understood that any or other suitable speed to allow an appropriate level of accuracy and/or sufficient level of information during navigation through a patients anatomy can be used. Thus, using the high-speed imaging capabilities of the CT machine in conjunction with the kit described herein, the surgeon is able to pierce the patient's anatomy and the target Tavlov cyst as described below.

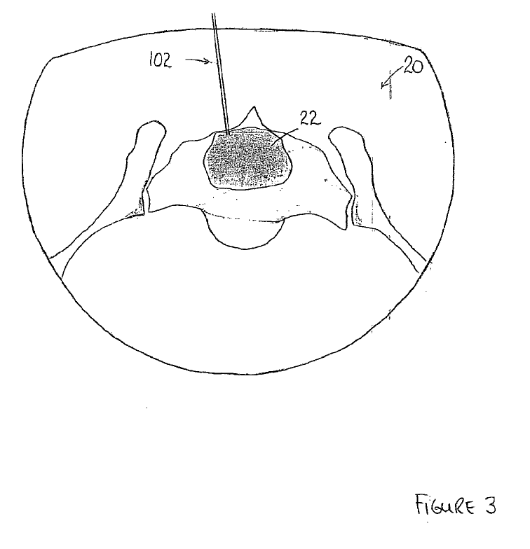

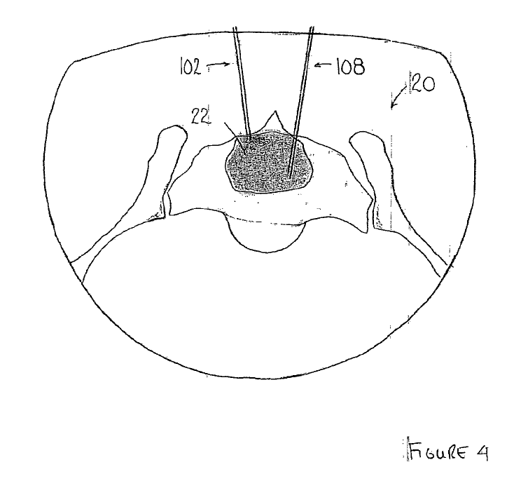

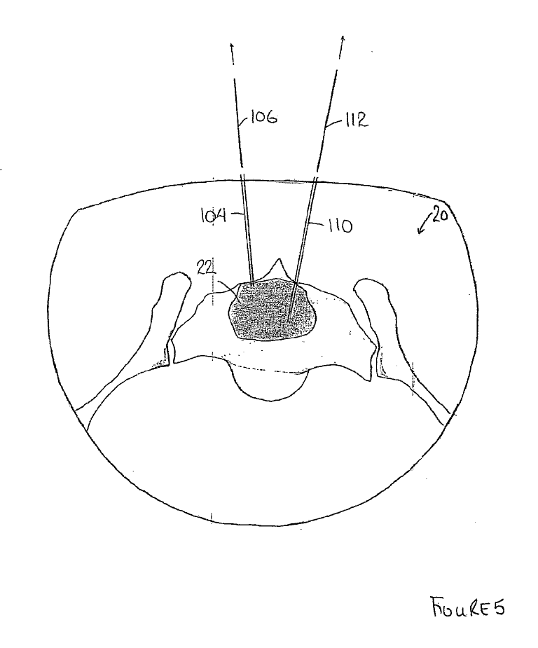

The use of the kit 100 to perform the method will now be explained. Referring back to FIG. 1, a patient undergoes a pre operative diagnostic CT (Toshiba Aquillion 16 slice MDCT), and the level/trajectory providing the best access to the cyst through the thinnest overlying bone is chosen. The patient's back is prepped in the usual sterile fashion as known in the art, and local anaesthesia is infiltrated into the skin, fat, muscles, and other tissues overlying the bony tissue to be pierced. As shown in FIG. 3, the first needle assembly 102 is advanced into the cyst. This first needle assembly 102 is placed superficially, near the cyst apex. Next, with the first needle assembly 102 maintained in position, the second needle assembly 108 is advanced deep into the cyst, as shown in FIG. 4. The stylets 106, 112 are then removed from the needle assemblies 102, 108, leaving the hollow trocars 104, 110 in position, as shown in FIG. 5. Through the deeper positioned second trocar 110, fluid is aspirated from the interior of the cyst, as shown in FIG. 6.

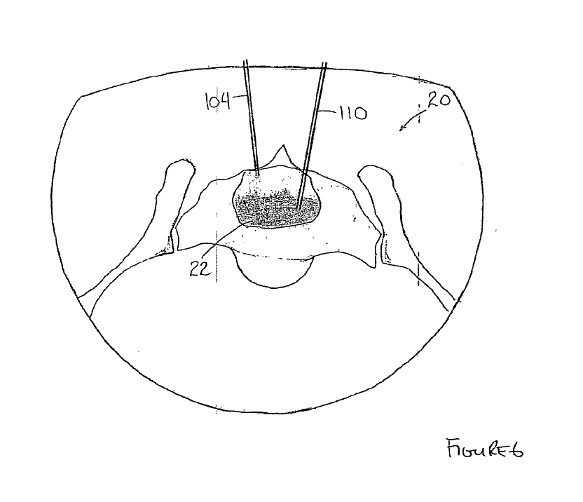

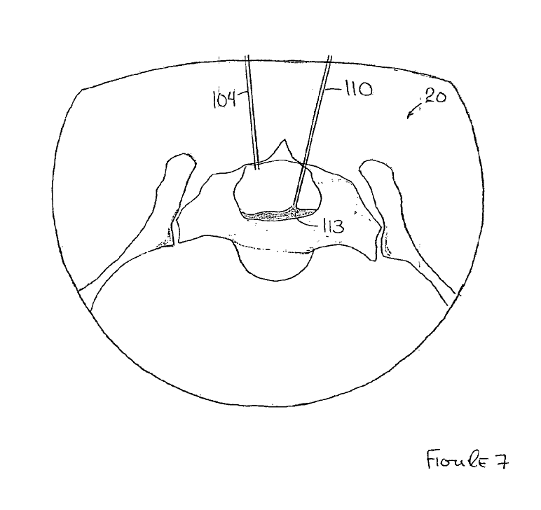

During the aspiration procedure, the first hollow trocar 104 of the first needle assembly 102 located near the apex of the cyst acts like a venting tube, allowing air to enter the cyst while fluid is removed through the second hollow trocar 110 of the second needle assembly 108. During the aspiration phase of the method, the air-fluid level is monitored intermittently under CT fluoroscopy for evidence of rapid cyst refilling, indicative of a wide necked cysts demonstrating CSF ingress. Such a scenario would require surgical repair prior to any further treatment, if necessary, for example to correct CSF leakage. With no indication of CSF ingress (e.g. stable air-fluid level), a tissue sealant (e.g. fibrin sealant) 113 is injected, into the air-containing cyst cavity through the hollow trocar 110 of the second needle assembly 108, with air escaping from the cavity through the first trocar 104, as shown in FIG. 7. The injection of the tissue sealant is stopped once the air-containing cavity of the cyst appears filled on CT fluoroscopy, as shown in FIG. 8. A filled cavity generally coincides with reflux of tissue sealant through the first hollow trocar 104 placed near the apex of the cyst. After filling, both the first and second hollow trocars 104, 110 are withdrawn, and the puncture sites suitably treated to prevent infection. For example, the puncture sites can be covered with an antibiotic ointment and a sterile dressing.

At any point during the procedure described above, the needle holder 114, or alternatively a plurality of needle holders can be used to hold and/or manipulate the trocars, as deemed necessary during the procedure.

To facilitate proper imaging of the tissue sealant during injection into the cyst cavity, the tissue sealant is formulated with a radio-opacity contrast component to increase its radio-opacity. For example, the tissue sealant could be formulated to include high atomic weight radio opaque materials, such as tantalum, tungsten or iodine. It will be appreciated however that those skilled in the art may choose to implement other physiologically acceptable radio-opacifiers to increase the radio-opacity of the tissue sealant. The radio-opacity of the tissue sealant could also be increased by introducing gas bubbles into the sealant. The incorporation of gas bubbles can be accomplished a number of ways, for example by shaking the sealant, thereby inducing foam formation, or by gas perfusion using, for example, a low density gas. A further option would be to incorporate fat into the sealant, as the contrast between the fat and tissue sealant enables visualization during imaging.

The use of a tissue sealant with increased radio-opacity allows for visualization under CT-guided fluoroscopy, permitting greater control during injection into the cyst cavity. The tissue sealant can also be used to seal CSF leaks after surgery, after spinal surgery, after Lumbar puncture, etc. The addition of a radio-opacity component to the tissue sealant improves visualization, enabling the surgeon to detect placement and migration of sealant during use. Proper visualization reduces the likelihood of improper placement or migration into non-target areas, such as for example the subarachanoid space or around the nerve roots, which can lead to inflammation, and potentially aseptic meningitis.

The two-needle technique was developed, at least partly, to help address the severe procedural pain arising from pressure variation in the cyst during treatment. The two-needle technique can markedly reduced the level of discomfort experienced by patients, and can greatly reduced the need for conscious sedation. At the same time, this technique can also allow increasing the amount of injected tissue sealant, which is believed to be a critical factor in improving the clinical outcome. The purpose of the needle placed superficially is to attenuate any pressure or volume changes within the cyst. Whereas it was previously found that the volumes of cerebrospinal fluid or tissue sealant that could be aspirated or injected were limited by patient pain, using the two-needle technique, the cyst can be substantially drained and filled without causing the patient pain until the maximum volume of the cyst is reached. For example, using this technique, as much as 16 millilitres of CSF have been aspirated from Tarlov cysts located at the S2-S3 level, in a virtually pain-free fashion under CT-guided fluoroscopy guidance. Filling of the cyst with tissue sealant is considered appropriate until the patient starts developing her/his typical symptoms or pain. These findings, which likely indicate cyst distension, are usually synchronous with reflux of the tissue sealant through the needle hub.

In addition to reduced pain for the patient, the two-needle method described above is easier to implement and considerably safer than open surgery which has a higher failure rate, and takes months to recover from. A greater precision and accuracy can be attained using the above described method. As indicated, the tools are specially constructed with radiolucent materials so as to reduce obstructive beam hardening artefacts during real-time visualization, while the tissue adhesive used to fill the cyst cavity is augmented with a radio-opacifier to improve visualization during injection.

An additional benefit of this procedure is the elimination of the need for myelographic contrast injection. If the cyst is wide necked and communicates with the CSF space, the air-fluid level changes rapidly as the cyst refills, in which instances tissue sealant is not injected until after surgical repair, if needed.

In general, there are three treatment paradigms:
good candidates with narrow necked cysts who go straight to tissue sealant injection under CT fluoroscopy using the above-described two-needle method;
surgical candidates with wide necked cysts who go to surgical repair; and
candidate requiring further investigation—they receive a CT myelogram with early and delayed imaging, to examine the cyst neck, and then are treated in the appropriate way according to either of group 1 or 2 above.

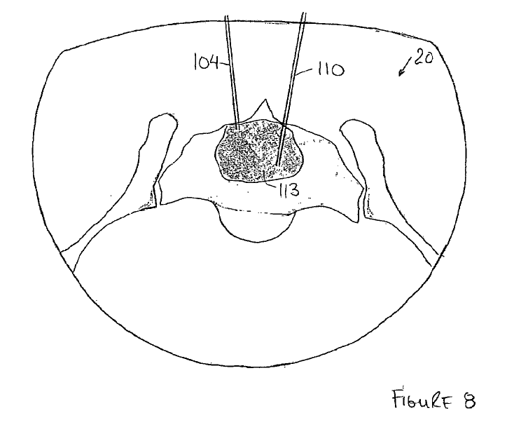

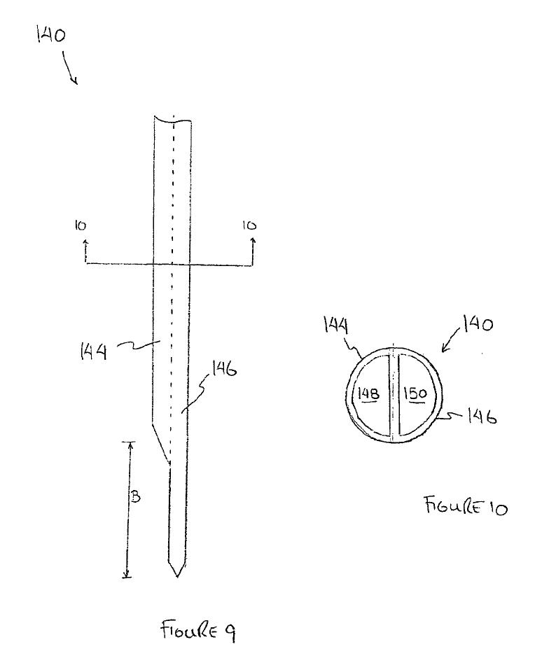

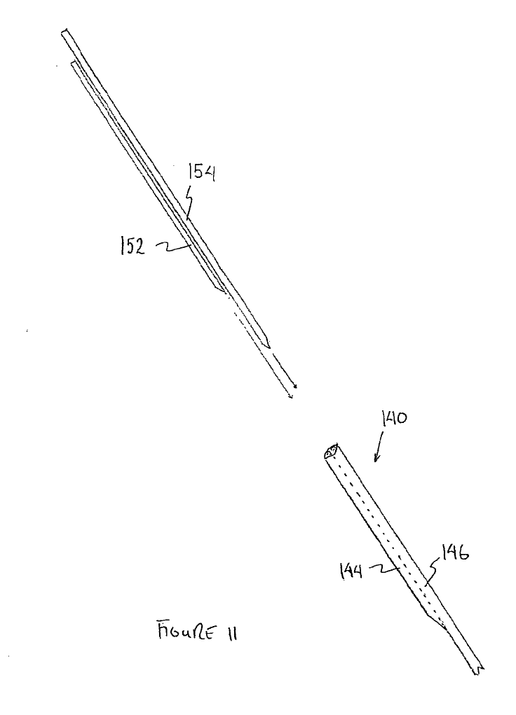

While the embodiment discussed above makes reference to two separate needles, it is conceivable to provide an alternate arrangement that provides a first venting lumen, and a second working lumen, and wherein the two lumens are provided in a single instrument. For example, in a first alternate embodiment, the first and second lumens could be provided in a side-by-side arrangement, as shown in the double-lumen needle assembly 140 of FIG. 9. As shown, the distal end of the double-lumen needle 140 is stepped, such that the first lumen 144 terminates above or short of the second lumen 146. The distance B defining the difference between the ends of the first and second lumens can be anywhere from 0.5 cm to 3 cm, but is preferably greater than 0.5 cm and less than 3 cm, and is more preferably approximately 1 cm. With this configuration, the first lumen 144 provides the venting function, while the second lumen 146 is used for aspiration and injection of the tissue sealant. As shown in cross-section in FIG. 10, the double-lumen needle 140 is generally cylindrical, with two opposing generally semi-circular channels 148, 150. As shown in FIG. 11, each semi-circular channel can be provided with a respective stylet 152, 154 for use during piercing and positioning within the target anatomy. Once in position, the stylets can be removed, and the double lumen needle used as described above, as shown in FIG. 12.

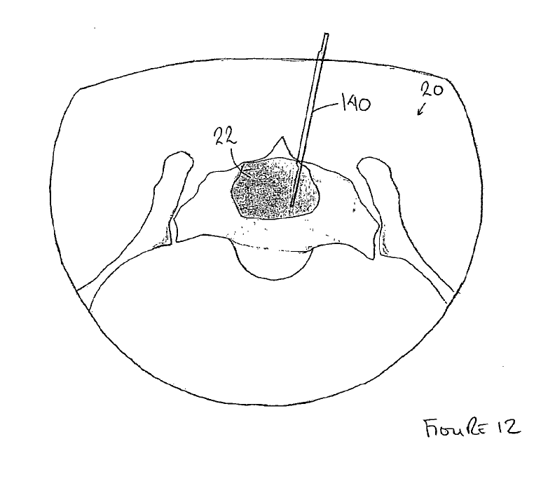

A further alternate embodiment is presented in FIG. 13 in which the distal end of the double-lumen needle assembly 160 can be configured with a vent hole 162 located along the needle shaft 164, on the side enclosing the first lumen 166. To prevent the ingress of tissue debris during piercing, the first lumen 166 and the second lumen 168 can be configured to receive respective stylets (not shown). The spacing of the vent hole 162 from the distal tip 170 can be anywhere from 0.5 cm to 3 cm, but is preferably greater than 0.5 cm and less than 3 cm, and is more preferably approximately 1 cm.

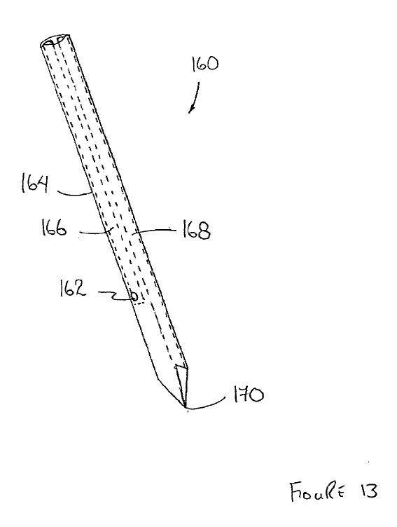

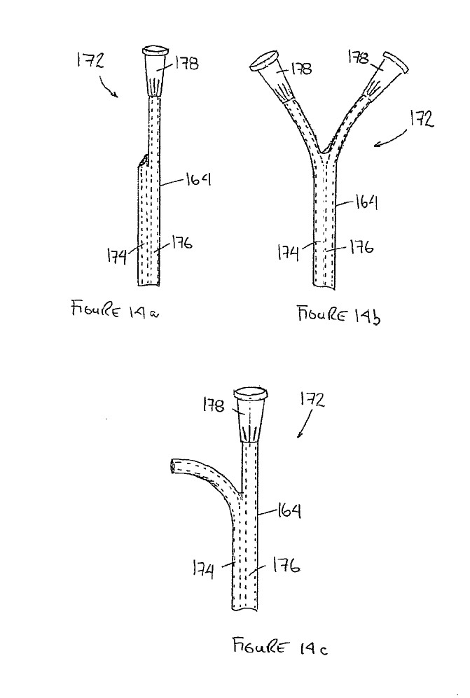

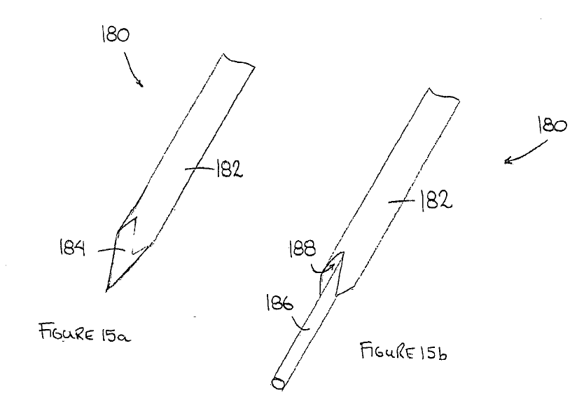

For a double-lumen needle assembly having the side-by-side configuration described above, the proximal end 172 of the needle shaft 164 can take on a variety of different configurations, as shown in FIGS. 14a, 14b and 14c. Each of the configurations presents a first lumen 174 for venting, and a second lumen 176 for aspiration/injection.

In instances where the proximal end 172 of the double-lumen needle assembly is shaped (non-linear), such as the Y-shaped configuration of FIG. 14b, the stylets used to prevent ingress of tissue debris into the lumen during piercing are preferably made from a flexible material.

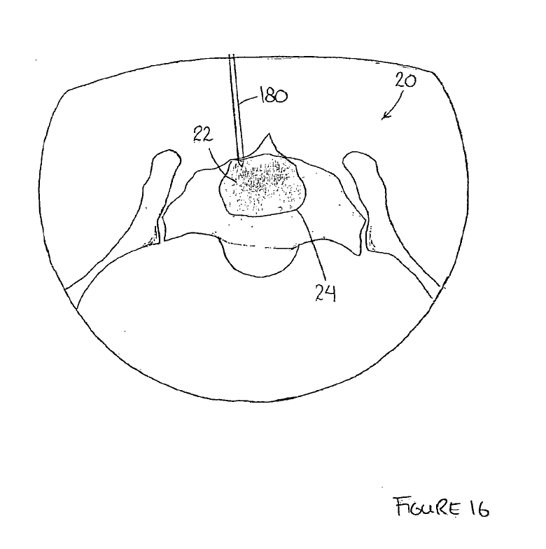

A further alternate embodiment is the co-axial arrangement of the first and second lumens, as shown in FIGS. 15a (readied for piercing) and 15b (readied for aspiration/injection). The arrangement shown in FIG. 15a is largely the same as either of the first or second needle assemblies used above in the two-needle approach. As shown, the needle assembly 180 comprises a hollow trocar 182 and a stylet 184 that is co-axially received through the hollow trocar 182. In one assembled piercing state as shown in FIG. 15a, the distal end of the trocar 182 and the tip of the stylet 184 form a contiguous tip for piercing through tissue. The contiguous tip can take on any variety of configurations, such as a three-sided point, a round arrow-head, or any other suitable arrangement suitable for piercing the intended tissue. In the arrangement shown in FIG. 15b, the needle assembly 180 has had the stylet 184 removed, and a smaller gauge needle 186 has been inserted into the trocar 182, defining an annulus 188 between the smaller gauge needle 186 and the inside wall of the hollow trocar 182. An example of this arrangement would have a 13 gauge trocar 182 and an 18 gauge needle 186. It will be appreciated, however, that a variety of gauge arrangements could be used so long as an annulus is created between the smaller gauge needle and the inside wall of the hollow trocar. In the above described co-axial arrangement defining annulus 188, the assembly is readied for aspiration and injection of the cyst.

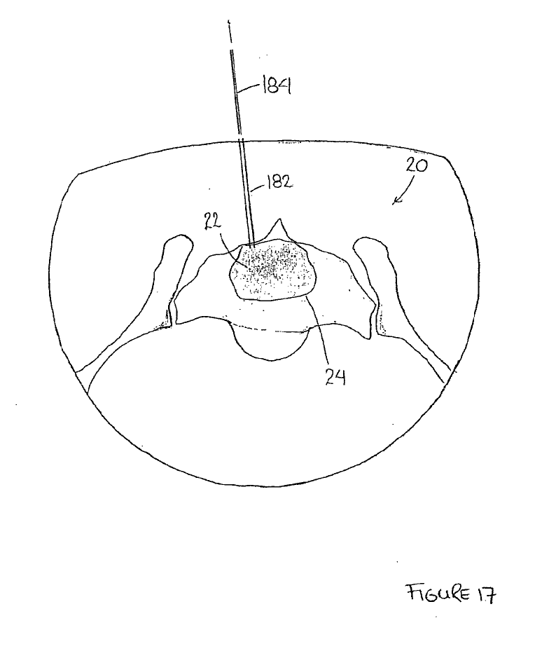

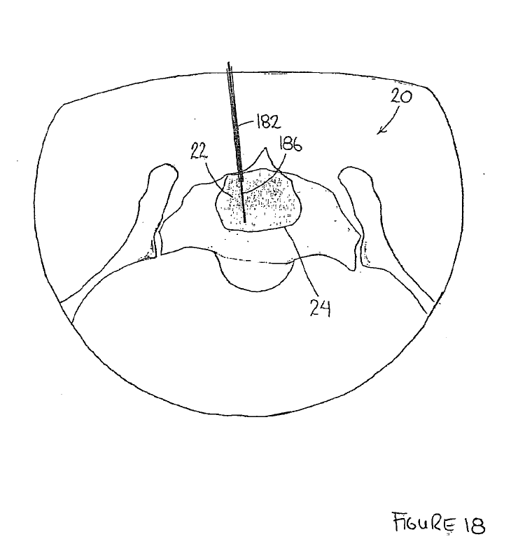

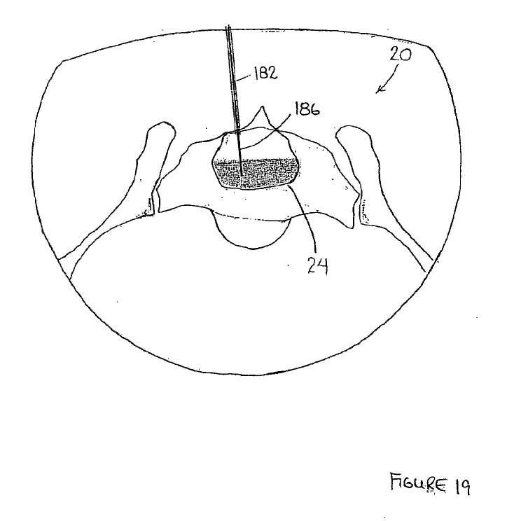

In use, as described above with respect to the two-needle approach, the needle assembly 180 is first advanced into the cyst. The needle assembly 180 is placed superficially, with the distal end positioned near the cyst apex, as shown in FIG. 16. The stylet 184 is then removed from the needle assembly 180, leaving the hollow trocar 182 in position, as shown in FIG. 17. Through the hollow trocar 182, a smaller gauge needle 186 is passed such that the distal end of the needle 186 is positioned deeper within the cyst, as shown in FIG. 18.

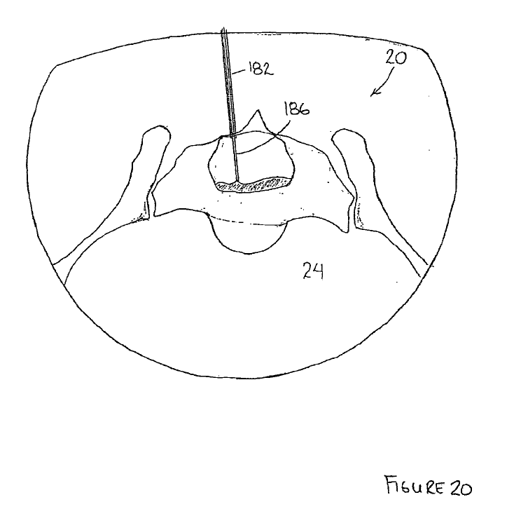

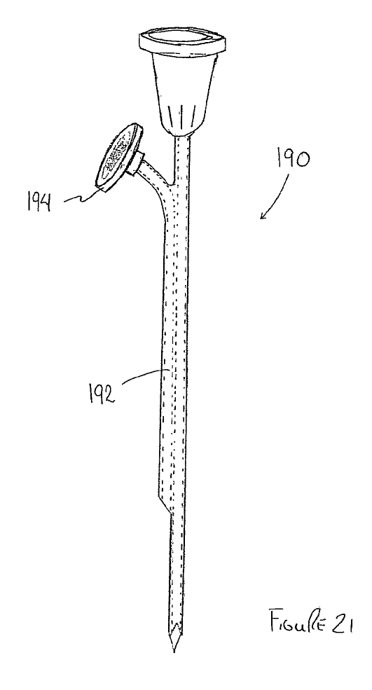

During the aspiration procedure, the annulus 188 located near the apex of the cyst acts like a venting tube, allowing air to enter the cyst while fluid is removed through the smaller gauge needle 186, as shown in FIG. 19. As during the two-needle approach, during the aspiration phase of the method, the air-fluid level is monitored intermittently under CT fluoroscopy for evidence of rapid cyst refilling, indicative of a wide necked cysts demonstrating CSF ingress. With no indication of CSF ingress (e.g. stable air-fluid level), a tissue sealant (e.g. fibrin sealant) 113 is injected, into the air-containing cyst cavity through the smaller gauge needle 186, with air escaping from the cavity through the annulus 188, as shown in FIG. 20. The injection of the tissue sealant is stopped once the air-containing cavity of the cyst appears filled on CT fluoroscopy. A filled cavity generally coincides with reflux of tissue sealant through the annulus 188 placed near the apex of the cyst. After filling, the needle 186 and hollow trocar 182 are withdrawn, and the puncture site suitably treated to prevent infection. For example, the puncture sites can be covered with an antibiotic ointment and a sterile dressing.

In a further alternate embodiment, as shown in FIG. 21, the first lumen 192 of the needle assembly 190 providing the vent function can be provided with a filter 194 to avoid contamination of the aspirated/treated cyst. The filter mesh or porosity should be such that effective air exchange can occur so as to allow the requisite reduction in pressure variations in the cyst, while preventing dust and other foreign particles from entering the cyst during treatment. It is presently preferable to locate the filter in the region of the proximal end of the first lumen 192. In addition, it is presently preferable that the filter be removable, and readily exchangeable when necessary.

In the various embodiments described above, to facilitate attachment of the needle assembly to a syringe or any other medical instrumentation, the proximal ends can be configured with suitable connectors. In the double-lumen needle assemblies shown in FIGS. 14a, 14b and 14c, the second lumens in each of FIGS. 14a, 14b and 14c, and the first lumen in FIG. 14b is shown with a Luer-lock connector 178. Other suitable connectors that could be implemented include bayonet-style connectors, and threaded connectors. One skilled in the art may use further alternate connectors as deemed suitable and applicable for a particular situation. Further, in instances where the proximal end of the needle assembly takes the form of a trocar handle, the handle could be configured with the aforementioned suitable connectors, thereby facilitating their connection to syringes or other medical instrumentation.

In addition, the needle assemblies can be further configured with handles or attachments for handles or other devices to allow the surgeons to establish a secure grip during use, such as during the piercing of the patient's anatomy.

The alternate embodiments described above (e.g. the double-lumen and co-axial configurations) are, presently, preferably made of a material that is hard enough and/or rigid enough to effect the desired piercing and travelling through tissue, but is also made from a generally radiolucent material with a radioopacity density such that the components create a reduced level of beam hardening artefacts when placed in the imaging beam. Suitable materials can include, but are not limited to, certain plastics (e.g. PEEK), carbon fiber and Inconel metals.

While only specific combinations of the various features and components of the present invention have been discussed herein, it will be apparent to those of skill in the art that desired subsets of the disclosed features and components and/or alternative combinations of these features and components can be utilized, as desired. For example, while the embodiments discussed herein refer to CT machines, it is to be understood that the teachings herein can be applied to any type of imaging machine capable of generating substantially real time images, such as machines based computerized tomography (“CT”), magnetic resonance (“MR”), or X-Ray. In addition, while the two needle assemblies being used have been generally described as being of the same gauge, it is entirely conceivable to conduct the method using needle assemblies of different gauges. For example, the needle assembly being used to vent the cyst can be of a smaller gauge, or quite possibly a larger gauge compared to the working needle assembly used for aspiration/injection. The same applies to the double lumen needles described above where the two side-by-side lumens need not be of the same gauge. In addition, while it is presently preferred to construct the various components using radiolucent materials, further embodiments can have only portions of the components or select components being made from radiolucent materials.

The above-described embodiments of the invention are intended to be examples of the present invention and alterations and modifications may be effected thereto, by those of skill in the art, without departing from the scope of the invention which is defined solely by the claims appended hereto.

1. A method for aspirating and treating a cyst comprising:
piercing a cyst with a first needle assembly until a distal tip of said first needle assembly is positioned in the region of the apex of the cyst, said first needle assembly comprising a first trocar and a first stylet;
piercing the cyst with a second needle assembly until a distal tip of said second needle assembly is positioned towards the far side of the cyst generally opposite said apex, said second needle assembly comprising a second trocar and a second stylet;
removing said first and second stylets and aspirating the contents of the cyst through said second trocar, said first trocar providing a vent to neutralize pressure variations within said cyst;
injecting a tissue sealant into the aspirated cyst through said second trocar.

2. The method of claim 1, wherein at least one of said first needle assembly and said second needle assembly is comprised of a substantially radiolucent material.

3. The method of claim 1, wherein at least one of said first needle assembly and said second needle assembly is comprised of carbon fibre.

4. The method of claim 1, wherein at least one of said needle assemblies is maintained in position using a needle holder.

5. The method of claim 1, wherein said tissue sealant is augmented so as to increase its radio-opacity during imaging.

6. The method of claim 1, wherein said tissue sealant is augmented with at least one of tantalum, tungsten and iodine to increase its radio-opacity during imaging.

7. A method of aspirating and treating a cyst comprising:
aspirating fluid from the cyst through a first lumen;
venting the cyst using a second lumen, so as to stabilize pressure variations within the cyst; and
injecting a tissue sealant into the aspirated cyst using the second lumen, while continuing to vent the cyst using said first lumen.

8. The method of claim 7, wherein said first lumen is provided by a first needle assembly, and wherein said second lumen is provided by a second needle assembly.

9. The method of claim 7, wherein said first lumen and said second lumen are provided on a single instrument.

10. The method of claim 9, wherein said first lumen and said second lumen are provided in a side-by-side arrangement, and wherein a distal opening of said first lumen is positioned approximately 0.5 cm to 3.0 cm above a distal opening of said second lumen.

11. The method of claim 9, wherein said first lumen and said second lumen are provided in co-axial arrangement, wherein said second lumen is defined by a needle that passes through said first lumen, such that said second lumen takes on a generally annular configuration.

12. The method of claim 7, wherein at least one of said first and second lumens are defined by substantially radio-lucent material.

13. The method of claim 7, wherein at least one of said first and second lumens are defined by respective first and second needle assemblies comprised of carbon fibre.

14. The method of claim 7, wherein said tissue sealant is augmented to increase its radio-opacity during imaging.

15. The method of claim 7, wherein said tissue sealant is augmented with at least one of tantalum, tungsten and iodine to increase its radio-opacity during imaging.

16. A kit of parts for cyst aspiration and treatment, said kit comprising:
a first needle assembly comprising a first trocar and a first stylet, a contiguous piercing tip for piercing through a patient's anatomy being formed when said stylet is assembled with said first trocar;
a second needle assembly comprising a second trocar and a second stylet, a contiguous piercing tip for piercing through a patient's anatomy being formed when said stylet is assembled with said second trocar;
a tissue sealant for injection into an aspirated cyst.

17. The kit according to claim 16, further comprising at least one needle holder for securing one of said first and second needle assemblies in position during piercing and subsequent use.

18. The kit according to claim 16, wherein at least one of said first needle assembly and said second needle assembly is comprised of a radio-lucent material

19. The kit according to claim 16, wherein at least one of said first needle assembly and said second needle assembly is comprised of carbon fibre.

20. The kit according to claim 16, wherein at least one of said needle assemblies is maintained in position using a needle holder.

21. The kit according to claim 16, wherein said tissue sealant is augmented so as to increase its radio-opacity during imaging.

22. The kit according to claim 16, wherein said tissue sealant is augmented with at least one of tantalum, tungsten and iodine to increase its radio-opacity during imaging.
